(19) United States
(12) Patent Application Publication
(10) Pub. No.: US 2022/0246312 A1
(43) Pub. Date: Aug. 4, 2022

(54) SYSTEM FOR ALTERING MEDICAL ENCOUNTERS BASED ON CULTURAL IDENTIFIERS

(71) Applicant: United Language Group, Inc., Minneapolis, MN (US)

(72) Inventors: Kristen Gail Giovanis, Minneapolis, MN (US); Nicholas Mcmahon, Portland, OR (US); Stephen Wade Torgeson, St. Paul, MN (US)

(21) Appl. No.: 17/549,581

(22) Filed: Dec. 13, 2021

Related U.S. Application Data
(60) Provisional application No. 63/143,268, filed on Jan. 29, 2021.

(51) Int. Cl. G16H 80/00; G06F 40/58

(52) U.S. Cl. CPC G16H 80/00; G06F 40/58

(57) ABSTRACT

This disclosure includes techniques for guiding a patient encounter using a computing device and cultural indicators. A computing device receives patient information for a first patient and determines, based at least in part on the patient information for the first patient and a model, cultural identifiers for the first patient. The computing device retrieves a set of encounter instructions for a first patient encounter for the first patient based on a patient encounter type of the first patient encounter for the first patient. The computing device develops an updated set of encounter instructions for the first patient encounter by altering the set of encounter instructions for the first patient encounter based on the cultural identifiers for the first patient. The computing device outputs, via an output component, the updated set of encounter instructions to guide the first patient encounter.

### PRIORITY INFORMATION

This application claims priority to U.S. Provisional Patent Application No. 63/143,268, filed Jan. 29, 2021, the entire contents of which are incorporated herein by reference.

### TECHNICAL FIELD

The disclosure relates to a system for directing medical encounters.

### BACKGROUND

Patients with Limited English Proficiency (LEP) are more likely than English-speaking patients to experience adverse outcomes within the healthcare system. LEP patients experiencing adverse events are more likely to be harmed, and harmed more seriously, than English-speaking patients. Moreover, adverse events experienced by LEP patients are more frequently caused by communication errors. A study published in the British Medical Journal found that adverse events that occur to LEP patients are 52% more likely to be the result of communication errors. Additionally, these adverse events are more serious for LEP patients. In another study, medication errors represented a significantly larger share (57%) of adverse events for LEP patients compared to English-speaking patients.

Risks for LEP patients are also compounded by low health literacy. LEP patients experience greater difficulty understanding instructions, including how to manage a condition, take their medications and prepare for a procedure. Acknowledging the inequities experience by LEP patients, systematic efforts have been made to require the availability of interpreters in hospital and clinical settings. However, the operational integration of onsite interpreters presents many practical challenges.

Additionally, several laws are routinely enforced in an effort to ensure that limited English proficiency (LEP) and deaf and hard-of-hearing patients are provided meaningful access to pertinent information surrounding their health care and well-being. These laws include: Section 1557 of the Affordable Care Act, Title VI of the Civil Rights Act, National Standards on Culturally and Linguistically Appropriate Services, Americans with Disabilities Act and the Hill Burton Act.

Section 1557 is the nondiscrimination provision of the Affordable Care Act (ACA). The law prohibits discrimination on the basis of race, color, national origin, sex, age, or disability in certain health programs or activities. The regulation builds on long-standing and familiar Federal civil rights laws including Title VI of the Civil Rights Act of 1964, Title IX of the Education Amendments of 1972, Section 504 of the Rehabilitation Act of 1973 and the Age Discrimination Act of 1975. Section 1557 extends nondiscrimination protections to individuals participating in any health program or activity any part of which received funding from the Department of Health and Human Services (HHS), any health program or activity that HHS itself administers, health insurance marketplaces and all plans offered by issuers that participate in those marketplaces. Section 1557 has been in effect since its enactment in 2010 and the HHS Office for Civil Rights has been enforcing the provision since it was enacted.

On May 13, 2016, the HHS Office for Civil Rights issued the final rule implementing Section 1557. The latest ruling emphasizes the importance of using a qualified medical interpreter and expressly prohibits the use of ad-hoc interpreters, including family members and other untrained bilingual individuals, barring extreme circumstances. The latest Section 1557 standards of the ACA, i.e., Nondiscrimination in Health Programs and Activities, were put into effect to increase access to health care, providing the same level of health-care services and coverage to all populations. Other regulations surrounding language access in healthcare include:

Title VI of the Civil Rights Act (1969) describes that services provided with the funding from the federal government must be delivered without regard to race, color, or national origin. National Standards on Culturally and Linguistically Appropriate Services (2001) include a set of 15 action steps intended to advance health equity, improve quality, and help eliminate health care disparities by providing a blueprint for individuals and health and health care organizations to implement culturally and linguistically appropriate services.

The Americans with Disabilities Act (1990) is a piece of civil rights legislation that prohibits discrimination and guarantees that people with disabilities have the same opportunities as everyone else to participate in the mainstream of American life—to enjoy employment opportunities, to purchase goods and services, and to participate in state and local government programs and services.

The Hill-Burton Act, enacted by Congress in 1946, encouraged the construction and modernization of public and nonprofit community hospitals and health centers. In return for receiving these funds, recipients agreed to comply with a “community service obligation,” one of which is a general principle of non-discrimination in the delivery of services. The Office of Civil Rights has consistently interpreted this as an obligation to provide language assistance to those in need of such services.

### SUMMARY

In general, the disclosure is directed to a system that assists providers and patients during a patient encounter, including encounters where certain cultural identifiers may lead to particular encounter instructions being beneficial as opposed to other patients who may be visiting the provider for similar encounters. For instance, based on certain patient information, the system may determine cultural identifiers for the patient using a model, such as a rule-based model or an artificial intelligence model. The system may alter a certain set of encounter instructions based on the determined cultural identifiers, either adding, removing, or changing certain questions or orders to suit particular medical characteristics that are more prevalent in patients that have those cultural identifiers. Cultural identifiers may be any descriptor of the patient that could influence how the medical encounter proceeds.

The disclosure is also directed to a system that assists in translations for the patient encounter when the patients are limited in their ability to speak the same language as the provider. When the system presents pre-translated questions to a patient, the patient response to those questions may lead to confusion or incorrect communications between the provider and the patient. If the system determines that the answers provided by the patient indicate that the encounter may be aided by real-time communication between the provider and the patient, the system may switch interfaces to a machine translation platform, where the provider may input free form questions that are translated for the patient. The system may translate the responses back to the language spoken by the provider, enabling the provider to obtain additional information important to the encounter.

In this way, the techniques of this disclosure effect particular treatments and prophylaxes for diseases and medical conditions by utilizing various data points to guide patient encounters. For instance, if a particular malady is more prevalent in groups of people with particular cultural identifiers, the system may automatically recognize when a patient fits into those particular cultural identifiers and alter a set of encounter instructions to ensure that the provider asks questions or performs tests to account for that particular malady. The system may further adjust wording so as not to offend the patient that may be sensitive to particular wording or add further information to discharge instructions. In any case, the system may directly alter how a provider treats a patient during a patient encounter by ensuring that risk factors inherent in patients with certain cultural identifiers are accounted for.

Furthermore, by automatically detecting when to switch platforms between pre-translation platforms (e.g., a platform where questions have previously been translated to account for cultural differences and regional dialects), machine translations (e.g., a real-time translation platform where questions and responses are input in one language and translated by one or more processors into a different language), and an interpreter platform (e.g., where a third-part interpreter is summoned to interpret a conversation between a patient and a provider), the techniques of this disclosure may further effect particular treatments and prophylaxes for diseases and medical conditions by ensuring that accurate and complete information is transferred between the provider and the patient. Furthermore, by automatically detecting when a platform switch is warranted, the techniques of this disclosure may reduce the amount of user inputs provided to the system, thereby improving the computing device overall by reducing the number of physical, logical, or otherwise user-generated inputs received and processed at the computing device that implements these techniques.

In one example, the disclosure is directed to a method for guiding a patient encounter. The method includes receiving, by one or more processors of a computing device, patient information for a first patient. The method further includes determining, by the one or more processors and based at least in part on the patient information for the first patient and a model, one or more cultural identifiers for the first patient. The method also includes retrieving, by the one or more processors, a set of one or more encounter instructions for a first patient encounter for the first patient based on a patient encounter type of the first patient encounter for the first patient. The method further includes developing, by the one or more processors, an updated set of one or more encounter instructions for the first patient encounter by altering the set of one or more encounter instructions for the first patient encounter based on the one or more cultural identifiers for the first patient. The method also includes outputting, by the one or more processors and via an output component, at least a portion of the updated set of one or more encounter instructions to guide the first patient encounter.

In another example, the disclosure is directed to a system that includes data store configured to store at least an artificial intelligence model, patient information for a plurality of patients, and a plurality of sets of one or more encounter instructions for patient encounters. The system further includes an output component. The system also includes one or more processors configured to receive patient information for a first patient. The one or more processors are further configured to determine, based at least in part on the patient information for the first patient and an artificial intelligence model, one or more cultural identifiers for the first patient. The one or more processors are also configured to retrieve, from the data store, a first set of one or more encounter instructions for a first patient encounter for the first patient based on a patient encounter type of the first patient encounter for the first patient. The one or more processors are further configured to develop an updated set of one or more encounter instructions for the first patient encounter by altering the first set of one or more encounter instructions for the patient encounter based on the one or more cultural identifiers for the first patient. The one or more processors are also configured to output, via the output component, at least a portion of the updated set of one or more encounter instructions to guide the first patient encounter.

In another example, the disclosure is directed to a method for assisting a patient encounter. The method further includes outputting, by one or more processors of a computing device and via an output component, a human language translation of a first encounter instruction of a set of one or more encounter instructions to guide a first patient encounter with a first patient, wherein the translation is a pre-defined translation. The method also includes receiving, by the one or more processors, a first patient response to the first encounter instruction. The method further includes determining, by the one or more processors, and based at least in part on one or more characteristics of the first patient response, to switch from a pre-defined translation platform to a machine translation platform. The method also includes receiving, by the one or more processors, an indication of user input comprising a supplemental instruction input by the provider. The method further includes outputting, by the one or more processors and via the output component, a machine translation of the supplemental instruction during the first patient encounter.

In another example, the disclosure is directed to a method for performing any of the techniques described herein.

In another example, the disclosure is directed to a device configured to perform any of the techniques described herein.

In another example, the disclosure is directed to an apparatus comprising means for performing any of the techniques described herein.

In another example, the disclosure is directed to a non-transitory computer-readable storage medium having stored thereon instructions that, when executed, cause one or more processors of a computing device to perform any of the techniques described herein.

In another example, the disclosure is directed to a system comprising one or more computing devices configured to perform any of the techniques described herein.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example environment in which a computing device may guide a patient encounter, in accordance with one or more techniques described herein.

FIG. 2 is a block diagram illustrating a more detailed example of a computing device configured to perform the techniques described herein.

FIG. 3 is a conceptual diagram illustrating a model and a plurality of data sources that may be used to train a model in accordance with one or more techniques described herein.

FIG. 4 is a flow diagram illustrating an example platform switching process in accordance with one or more techniques described herein.

FIG. 5 is a flow diagram illustrating an example patient encounter in accordance with one or more techniques described herein.

FIG. 6 is a flow diagram illustrating an example patient encounter in accordance with one or more techniques described herein.

FIG. 7 is a flow diagram illustrating an example platform switching process in accordance with one or more techniques described herein.

### DETAILED DESCRIPTION

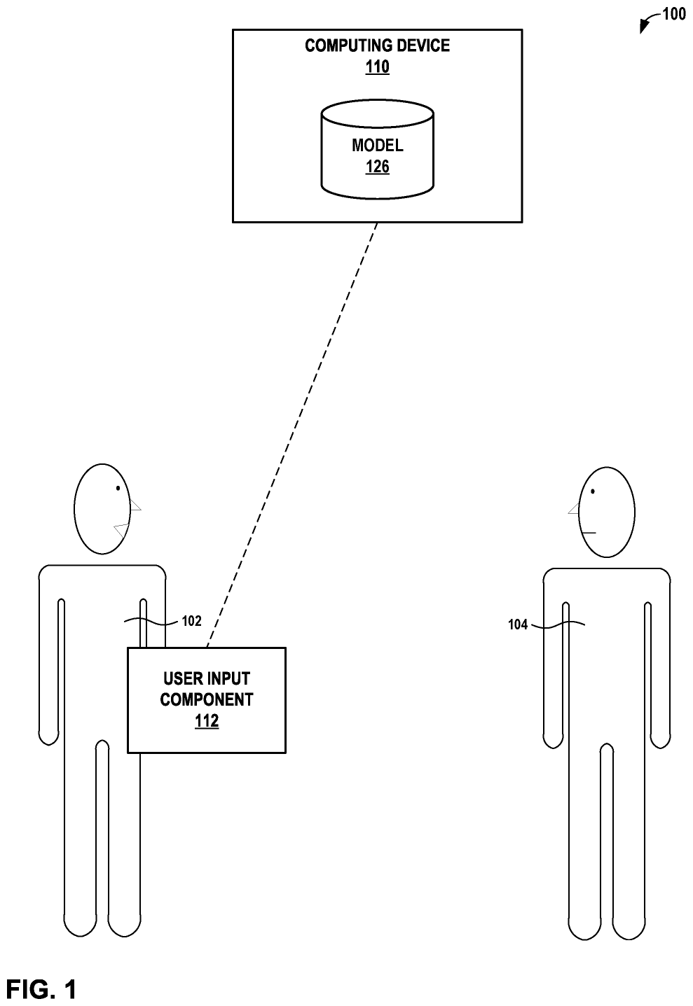

FIG. 1 is a conceptual diagram illustrating an example environment 100 in which computing device 110 may guide a patient encounter, in accordance with one or more techniques described herein. As shown in FIG. 1, computing device 110 includes model 126, which may be a rule-based model or an artificial intelligence model that continues to grow and develop as new data is introduced by computing device 110. Computing device 110 may be operatively connected to user input component (UIC) 112, which may be either a physical component of computing device 110 or a separate component configured to communicate with computing device 110 through either a wired or wireless connection.

Computing device 110 may communicate with UIC 112 to output various encounter instructions to guide a patient encounter. For instance, as shown in FIG. 1, user 102 may be a provider, and user 104 may be a patient. Computing device 110 may output various questions, orders, directions, or talking points for user 102 to discuss with user 104 for a certain type of patient encounter. This includes images, text, videos, translations, or audio instructions that user 102 can go over with user 104.

In other instances, user 102 may be the patient and user 104 may be an intake specialist. In such instances, user 102 may have UIC 112 and may read the instructions to input various information normally received during the intake process (e.g., purpose of visit, name, identifying information, etc.). In this way, whether it is guiding the provider or guiding the patient, computing device 110 may utilize UIC 112 to guide the patient encounter to ensure that the patient receives proper treatment.

In guiding the encounter, computing device 110 may utilize model 126. Model 126 may be designed to change a typical set of patient instructions based on one or more cultural indicators or identifiers (e.g., a country of origin, a preferred language, a region of origin, a religion, a time of year, a day of a week, an age, a family descendance, a birth gender, a personal gender, a sexual orientation, a skin color, and a residence location, among other things). For a certain type of patient encounter, computing device 110 and model 126 may presume that any of these cultural indicators indicate an atypical (e.g., different in a statistically significant manner) issue that may be present for that patient, such as an abnormally high chance of an additional disease being present in the patient based on health factors of the population of those with those same cultural indicators. These cultural indicators may also indicate that the phrasing of certain questions should be altered to avoid controversial terminology (e.g., obtaining personal pronouns for transgendered patients, or avoiding specific words that are offensive in certain religions). In this way, computing device 110 may ensure that, regardless of what type of encounter the patient is being seen for, cultural identifiers are recognized and accounted for when developing treatment plans and procedures for patients, including those with limited capabilities to speak the same language as the provider or intake specialist.

In the United States, federal regulations mandate language access through qualified interpretation and translation services for Limited English Proficient persons. Current solutions to provide services are supported by over-the-phone interpretations, on-site interpretation, video remote interpretation, and written translation services.

The interpreter role in these existing services it to be impartial. The interpreter cannot help the patient or clinician and should only interpret the words each person uses.

There are several challenges that the existing services fail to resolve. For instance, the interpreters are underutilized due to the cost model to service LEP patients. Due to it being an unfunded mandate, the cost burden is most often on the provider, therefore the desire is to keep interactions as brief as possible. Current services remain underutilized for certain interactions, such as admissions or in room care. There are inefficiencies and health risk due to wait time to access an interpreter, as well as insufficient access to small language population resources that cause interpreters to be underutilized. Furthermore, there is a lack of cultural competency of clinicians which results in misunderstandings and lack of patient engagement. Additionally, health literacy challenges of patients should prompt additional comprehension or health strategy, but currently do not. Adding to the stress of the clinical event, LEP patients are often left to use non-verbal communication to get by or, at times, are provided with informal interpretation by non-qualified staff.

The techniques described herein seek to fill this gap by developing proprietary technology that can provide a seamless patient experience for LEP patients through a communications platform customized in their native language and culture. Though initially designed for those with LEP, these techniques can be extended to any cultural indication where the inherent differences in culture could lead to differentiation in how care should be provided to those patients, with those cultural indications including religion, gender (both personal and born), age, location, and skin color, among other things. The techniques described herein address many practical barriers hospitals and clinics experience in providing qualified interpreters and translations at important junctures. The communication platform described herein solves for many common barriers observed in the field. Examples for which the platform could provide solutions include situations where there is no interpreter at check-in/admissions, no interpreter available in the required language, no interpreter available for routine inpatient room check-ins, no interpreter available to communicate with LEP family members and caregivers, no interpreter available for non-critical communication such as room care, lack of available written content in language, or a lack of immediate translation of content.

The techniques described herein allow users to provide language access that is culturally specific and supported through four deliveries: translated content for common interactions, machine translations for content not included in pre-translated content, over the phone interpretation, and video-remote interpretation. The base system provides access to the four language access deliveries for users in healthcare setting with interaction specific pre-translated questions and responses.

In one instance, a clinic that implements the techniques described herein see an Arabic patient at clinical admissions. The admission's user is able to utilize the system to queue list of standard questions or statements for that interaction type in the Arabic language with pre-translated responses to select from. If responses result in a question that is not currently translated, the user and LEP can use the machine translation function to ask and answer question in the respective languages in a free-form manner. If neither the pre-translated content nor machine translation are effective and/or efficient, the user can select to utilize either over-the-phone interpretation (OPI) or video remote interpretation (VRI). For instance, based on the free-flow machine translation questions or responses, computing device 110 may prompt the user to connect to interpreter.

In addition to providing easy, immediate access to an interpreter in their native language, the communications platform described herein is supported by existing pre-translated content for common scenarios and Neural Machine Translation (NMT) capabilities to help translate and deliver content in the patient's native language.

Features of the communications platform described herein have also been designed to address common root causes of communication-based adverse events. For instance, when a patient uses family members, friends, or nonqualified staff as interpreters, the techniques described herein may utilize a touchscreen that provides visual cues to help patient communicate with clinical staff for routine in-patient requests. The techniques described herein further allow connection with a qualified interpreter using multiple modalities. The interpreter and the patient may communicate jointly and directly with clinical team. In these instances, family and friends are alleviated of the responsibility of ensuring accurate information is provided to the provider, instead making them available to engage and provide support. In instances when cultural beliefs and traditions are affecting patient care, the techniques described herein may be configured to the cultural cues common to identified LEP patient populations.

Additional artificial intelligence (AI) capabilities can also create new or modify questions within specific interactions. Using the example above with a patient at clinical admissions, the question ‘Do you have insurance?’ may be typed as a free form question a certain number of times after pre-translated translations or questions. Using this information, the AI in model 126 may generate the question as a pre-translated question and response within the interaction type.

Additional AI capabilities can also integrate important cultural cue data, or surfacing information customized to the unique cultures served in the identified patient population. These cues can be displayed for both the clinician and the interpreter. One example of such cues includes an Arabic patient at a hospital being discharged. Based on data, there may be a significant likelihood of emergency room readmission due to failure to follow medication instructions. The cultural cue or alert may suggest a clinician and/or interpreter request for the patient to reiterate verbally the instructions to ensure comprehension.

Another example could include a Hmong patient at hospital discharge. Based on demographic and/or patient data, model 126 could indicate this as a high social determinants of health (SDoH) risk. Model 126 may include a cultural cue to audit for SDoH and arrange post stay services (i.e. transportation, food).

Another example could include a Chinese patient during a medication reconciliation interaction. Based on data health literacy, model 126 could indicate that this interaction may be a challenge and output a cultural cue to utilize teach back interpretation methods to ensure accurate data collection. While these examples focus on countries of origin, it should be realized that any cultural indication described herein could cause similar adjustments to treatment procedures.

The communication platform described herein is further designed to fill existing gaps in the provision of language services within both hospital and clinic settings. The techniques described herein have the potential to fill operational gaps, improve efficiencies, reduce costs, and enhance outcomes among LEP patients (as well as other patients with medical tendencies due to their cultural identifiers).

In accordance with the techniques described herein, computing device 110 may receive patient information for a first patient. Computing device 110 may determine, based at least in part on the patient information for the first patient and model 126, one or more cultural identifiers for the first patient. Computing device 110 may retrieve a set of one or more encounter instructions for a first patient encounter for the first patient based on a patient encounter type of the first patient encounter for the first patient. Computing device 110 may develop an updated set of one or more encounter instructions for the first patient encounter by altering the set of one or more encounter instructions for the first patient encounter based on the one or more cultural identifiers for the first patient. Computing device 110 may output, via UIC 112, at least a portion of the updated set of one or more encounter instructions to guide the first patient encounter.

Further in accordance with the techniques described herein, computing device 110 may output, UIC 112, a human language translation of a first encounter instruction of a set of one or more encounter instructions to guide a first patient encounter with a first patient, with the translation being a pre-defined translation. Computing device 110 may receive a first patient response to the first encounter instruction. Computing device 110 may determine, based at least in part on one or more characteristics of the first patient response, to switch from a pre-defined translation platform to a machine translation platform. Computing device 110 may receive, at UIC 112, an indication of user input comprising a supplemental instruction input by the provider. Computing device 110 may output, via UIC 112, a machine translation of the supplemental instruction during the first patient encounter.

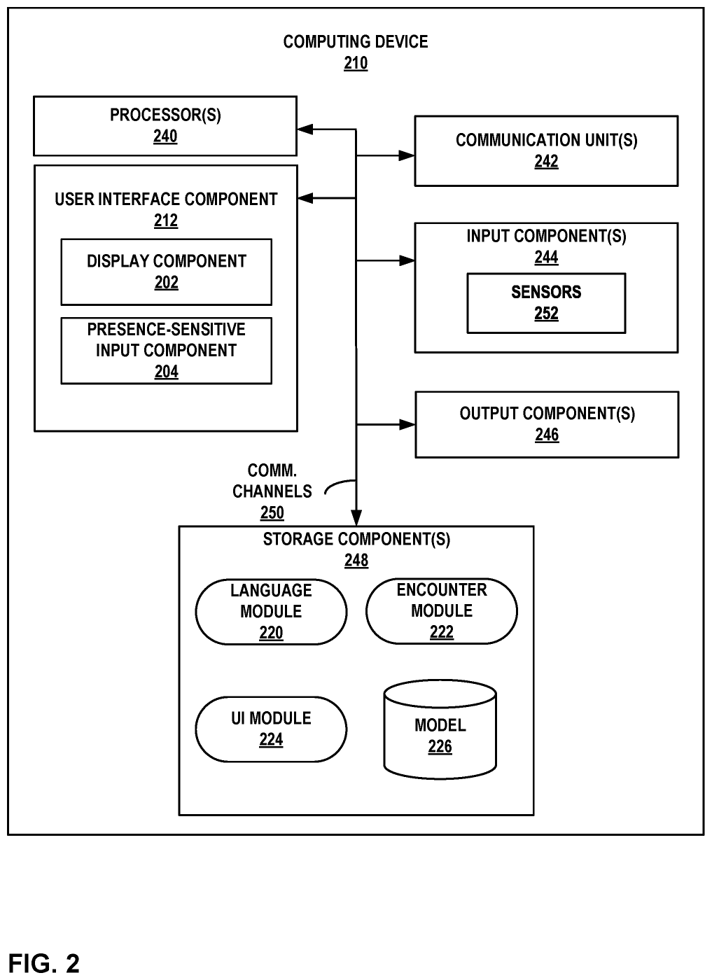

FIG. 2 is a block diagram illustrating a more detailed example of a computing device configured to perform the techniques described herein. Computing device 210 of FIG. 2 is described below as an example of computing device 110 of FIG. 1. FIG. 2 illustrates only one particular example of computing device 210, and many other examples of computing device 210 may be used in other instances and may include a subset of the components included in example computing device 210 or may include additional components not shown in FIG. 2.

Computing device 210 may be any computer with the processing power required to adequately execute the techniques described herein. For instance, computing device 210 may be any one or more of a mobile computing device (e.g., a smartphone, a tablet computer, a laptop computer, etc.), a desktop computer, a smarthome component (e.g., a computerized appliance, a home security system, a control panel for home components, a lighting system, a smart power outlet, etc.), a wearable computing device (e.g., a smart watch, computerized glasses, a heart monitor, a glucose monitor, smart headphones, etc.), a virtual reality/augmented reality/extended reality (VR/AR/XR) system, a video game or streaming system, a network modem, router, or server system, or any other computerized device that may be configured to perform the techniques described herein.

As shown in the example of FIG. 2, computing device 210 includes user interface component (UIC) 212, one or more processors 240, one or more communication units 242, one or more input components 244, one or more output components 246, and one or more storage components 248. UIC 212 includes display component 202 and presence-sensitive input component 204. Storage components 248 of computing device 210 include language module 220, encounter module 222, UI module 224, and model 226.

One or more processors 240 may implement functionality and/or execute instructions associated with computing device 210 to dynamically alter encounter instructions to create custom instructions for a patient encounter based on cultural indications of the patient. That is, processors 240 may implement functionality and/or execute instructions associated with computing device 210 to dynamically add, remove, or alter encounter instructions for a particular patient encounter using model 226 and one or more cultural identifiers for the patient.

Examples of processors 240 include application processors, display controllers, auxiliary processors, one or more sensor hubs, and any other hardware configure to function as a processor, a processing unit, or a processing device. Modules 220, 222, and 224 may be operable by processors 240 to perform various actions, operations, or functions of computing device 210. For example, processors 240 of computing device 210 may retrieve and execute instructions stored by storage components 248 that cause processors 240 to perform the operations described with respect to modules 220, 222, and 224. The instructions, when executed by processors 240, may cause computing device 210 to dynamically add, remove, or alter encounter instructions for a particular patient encounter using model 226 and one or more cultural identifiers for the patient.

UI module 224 may execute locally (e.g., at processors 240) to provide functions associated with managing a user interface that computing device 210 provides at UIC 212 for example, for facilitating interactions between a user of computing device 210 and UI module 224. In some examples, UI module 224 may act as an interface to a remote service accessible to computing device 210. For example, UI module 224 may be an interface or application programming interface (API) to a remote server that outputs (e.g., displaying) interface elements associated with the techniques described herein.

In some examples, language module 220 and encounter module 222 may execute locally (e.g., at processors 240) to provide functions associated with determining cultural indications and encounter instructions for a patient encounter. In some examples, language module 220 and encounter module 222 may act as an interface to a remote service accessible to computing device 210. For example, language module 220 and encounter module 222 may each be an interface or application programming interface (API) to a remote server that determines cultural indications and encounter instructions for a patient encounter.

One or more storage components 248 within computing device 210 may store information for processing during operation of computing device 210 (e.g., computing device 210 may store data accessed by modules 220, 222, and 224 and model 226 during execution at computing device 210). In some examples, storage component 248 is a temporary memory, meaning that a primary purpose of storage component 248 is not long-term storage. Storage components 248 on computing device 210 may be configured for short-term storage of information as volatile memory and therefore not retain stored contents if powered off. Examples of volatile memories include random access memories (RAM), dynamic random-access memories (DRAM), static random-access memories (SRAM), and other forms of volatile memories known in the art.

Storage components 248, in some examples, also include one or more computer-readable storage media. Storage components 248 in some examples include one or more non-transitory computer-readable storage mediums. Storage components 248 may be configured to store larger amounts of information than typically stored by volatile memory. Storage components 248 may further be configured for long-term storage of information as non-volatile memory space and retain information after power on/off cycles. Examples of non-volatile memories include magnetic hard discs, optical discs, flash memories, or forms of electrically programmable memories (EPROM) or electrically erasable and programmable (EEPROM) memories. Storage components 248 may store program instructions and/or information (e.g., data) associated with modules 220, 222, and 224 and model 226. Storage components 248 may include a memory configured to store data or other information associated with modules 220, 222, and 224 and model 226.

Communication channels 250 may interconnect each of the components 212, 240, 242, 244, 246, and 248 for inter-component communications (physically, communicatively, and/or operatively). In some examples, communication channels 250 may include a system bus, a network connection, an inter-process communication data structure, or any other method for communicating data.

One or more communication units 242 of computing device 210 may communicate with external devices via one or more wired and/or wireless networks by transmitting and/or receiving network signals on one or more networks. Examples of communication units 242 include a network interface card (e.g. such as an Ethernet card), an optical transceiver, a radio frequency transceiver, a GPS receiver, or any other type of device that can send and/or receive information. Other examples of communication units 242 may include short wave radios, cellular data radios, wireless network radios, as well as universal serial bus (USB) controllers.

One or more input components 244 of computing device 210 may receive input. Examples of input are tactile, audio, and video input. Input components 244 of computing device 210, in one example, includes a presence-sensitive input device (e.g., a touch sensitive screen, a PSD), mouse, keyboard, voice responsive system, camera, microphone or any other type of device for detecting input from a human or machine. In some examples, input components 244 may include one or more sensor components (e.g., sensors 252). Sensors 252 may include one or more biometric sensors (e.g., fingerprint sensors, retina scanners, vocal input sensors/microphones, facial recognition sensors, cameras) one or more location sensors (e.g., GPS components, Wi-Fi components, cellular components), one or more temperature sensors, one or more movement sensors (e.g., accelerometers, gyros), one or more pressure sensors (e.g., barometer), one or more ambient light sensors, and one or more other sensors (e.g., infrared proximity sensor, hygrometer sensor, and the like). Other sensors, to name a few other non-limiting examples, may include a heart rate sensor, magnetometer, glucose sensor, olfactory sensor, compass sensor, or a step counter sensor.

One or more output components 246 of computing device 210 may generate output in a selected modality. Examples of modalities may include a tactile notification, audible notification, visual notification, machine generated voice notification, or other modalities. Output components 246 of computing device 210, in one example, includes a presence-sensitive display, a sound card, a video graphics adapter card, a speaker, a cathode ray tube (CRT) monitor, a liquid crystal display (LCD), a light emitting diode (LED) display, an organic LED (OLED) display, a virtual/augmented/extended reality (VR/AR/XR) system, a three-dimensional display, or any other type of device for generating output to a human or machine in a selected modality.

UIC 212 of computing device 210 may be similar to UIC 112 of computing device 110 and includes display component 202 and presence-sensitive input component 204. Display component 202 may be a screen, such as any of the displays or systems described with respect to output components 246, at which information (e.g., a visual indication) is displayed by UIC 212 while presence-sensitive input component 204 may detect an object at and/or near display component 202.

While illustrated as an internal component of computing device 210, UIC 212 may also represent an external component that shares a data path with computing device 210 for transmitting and/or receiving input and output. For instance, in one example, UIC 212 represents a built-in component of computing device 210 located within and physically connected to the external packaging of computing device 210 (e.g., a screen on a mobile phone). In another example, UIC 212 represents an external component of computing device 210 located outside and physically separated from the packaging or housing of computing device 210 (e.g., a monitor, a projector, etc. that shares a wired and/or wireless data path with computing device 210).

UIC 212 of computing device 210 may detect two-dimensional and/or three-dimensional gestures as input from a user of computing device 210. For instance, a sensor of UIC 212 may detect a user's movement (e.g., moving a hand, an arm, a pen, a stylus, a tactile object, etc.) within a threshold distance of the sensor of UIC 212. UIC 212 may determine a two or three-dimensional vector representation of the movement and correlate the vector representation to a gesture input (e.g., a hand-wave, a pinch, a clap, a pen stroke, etc.) that has multiple dimensions. In other words, UIC 212 can detect a multi-dimension gesture without requiring the user to gesture at or near a screen or surface at which UIC 212 outputs information for display. Instead, UIC 212 can detect a multi-dimensional gesture performed at or near a sensor which may or may not be located near the screen or surface at which UIC 212 outputs information for display.

In accordance with one or more techniques of this disclosure, encounter module 222 receives patient information for a first patient. This could be either as part of an intake process where a provider gathers information from the patient, or could simply be that the first patient is a new patient for which no known information is present. This information receiving action could be as simple as the new patient indication or an indication of the spoken language of the first patient, or could be as complex as a full personal and/or medical history for the first patient. Any information-receiving step that could lead to a determination of cultural identifiers may be accomplished by encounter module 222.

Encounter module 222 may determine, based at least in part on the patient information for the first patient and model 226, one or more cultural identifiers for the first patient. The one or more cultural identifiers may be any one or more of a country of origin, a preferred language, a region of origin, a religion, a time of year, a day of a week, an age, a family descendance, a birth gender, a personal gender, a sexual orientation, a skin color, a residence location, or any other culturally descriptive information for the first patient that could influence their healthcare process and outcomes. In general, the cultural identifier could be any descriptor of the patient that could lead to particular medical issues being more prevalent or likely in that patient based solely on the fact that they have those cultural identifiers.

Encounter module 222 may retrieve a set of one or more encounter instructions for a first patient encounter for the first patient based on a patient encounter type of the first patient encounter for the first patient, such as from a data store in storage components 248. The patient encounter type may be any one or more of a patient intake process, a medical examination, a pharmaceutical consultation, a follow-up examination, a patient discharge, a patient admittance, in-room patient care, and an unplanned patient visit. Even further each of these types of encounters may be specific to a particular disease, ailment, medication, procedure, or purpose. In general, the set of one or more encounter instructions are a general set of instructions that a provider would typically perform for a particular patient's visit given the reason for their visit.

Encounter module 222 may develop an updated set of one or more encounter instructions for the first patient encounter by altering the set of one or more encounter instructions for the first patient encounter based on the one or more cultural identifiers for the first patient. Altering the set of one or more encounter instructions could include any one or more of adding a new encounter instruction to the set of one or more encounter instructions, removing an encounter instruction from the set of one or more encounter instructions, or changing content of an encounter instruction from the set of one or more encounter instructions. Each encounter instruction in the updated set of one or more encounter instructions may be one or more of a question to be asked to the first patient by the provider, an order to be given to the first patient by the provider, a procedure to be performed on the first patient by the provider, information to be gathered from the first patient by the provider, or medication to be given to the first patient by the provider. Ultimately, the updated set of one or more encounter instructions will be a patient-tailored set of steps to be performed by the provider (or information to be provided by the patient) based on the reason the patient is seeing the provider and based on cultural identifiers that may make the patient more prone to a particular issue during the course of that encounter.

UI module 224 may output, via UIC 112 or output components 246, at least a portion of the updated set of one or more encounter instructions to guide the first patient encounter. UI module 224 may output the updated set of encounter instructions either as the graphical text, videos, or images via a screen, audio via a speaker, or a hard copy of at least the portion of the updated set of one or more encounter instructions via a printer. In some instances, in outputting at least the portion of the updated set of one or more encounter instructions, language model 220 may translate at least the portion of the updated set of one or more encounter instructions, and UI module 224 may output the translation of at least the portion of the updated set of one or more encounter instructions, with the translation is based at least in part on the one or more cultural identifiers for the first patient. In this way, the patient is able to understand the goal of the encounter instruction and the provider may have a facilitated conversation with the patient as part of the encounter.

In one example of a first patient encounter, UI module 224 may output a translation of a first encounter instruction of the updated set of one or more encounter instructions. The particular language of the translation may be based on the one or more cultural identifiers for the first patient, as the preferred language, country of origin, or residence location, among other things, may be included in the cultural identifiers. The translation of the first encounter instruction may be a pre-translated version of the first encounter instruction presented over a pre-translation platform (e.g., a platform where pre-populated questions are provided translations that account for context and potential regional dialects to be the most accurate possible translation for the translation to be provided).

UI module 224 may receive a first patient response to the first encounter instruction, such as in the form of an indication of user input (e.g., typed or spoken word). In some instances, language module 220 may translate that patient response into the language spoken by the provider.

Language module 220 may determine, based at least in part on one or more characteristics of the first patient response, to switch from the pre-translation platform to a machine translation platform, or a platform where an electronic system predicts a best translation for terms and phrases between languages. These one or more characteristics of the first patient response may include one or more of a time it took for the first patient to provide the first patient response to the first encounter instruction (e.g., if the patient does not respond within a set amount of time, such as 3, 5, 8, or 10 seconds, or a time set to be longer and shorter), content of the first patient response (e.g., the patient saying “no”, the patient answering in a way that requires follow-up questions, or an indeterminate response such as “I don't know”), or the first patient response being an indication of silence (e.g., the patient does not respond at all). In some instances, encounter module 222 further makes this determination based on model 226 and the one or more cultural identifiers for the first patient.

In some instances, UI module 224 may issue a prompt for the provider to input a supplemental instruction. The supplemental instruction may be a free-for instruction provided by the provider, either via spoken word or typed text, that is not initially a part of the updated set of one or more encounter instructions, but is instead necessitated by the responses provided by the patient. Encounter module 222 may receive an indication of user input comprising the supplemental instruction input by the provider. Language module 220 may then perform a machine translation on the supplemental instruction to be into the language spoken by the patient, and UI module 224 may output, via UIC 212 or output components 246, the machine translation of the supplemental instruction during the first patient encounter.

In some instances, encounter module 222 may then receive a second patient response to the supplemental instruction. In some instances, this may lead to the provider being satisfied with the answer and switching back to the pre-translated platform and continuing the encounter based on the updated set of one or more encounter instructions. In other instances, encounter module 222 may determine, based at least in part on one or more characteristics of the second patient response, to prompt the provider to initiate contact with a human interpreter. The one or more characteristics of the second patient response may include one or more of a time it took for the first patient to provide the second patient response to the supplemental instruction (e.g., if the patient does not respond within a set amount of time, such as 3, 5, 8, or 10 seconds, or a time set to be longer and shorter), content of the second patient response (e.g., the patient saying “no”, the patient answering in a way that requires follow-up questions, or an indeterminate response such as “I don't know”), the second patient response being an indication of silence (e.g., the patient does not respond at all), or a number of patient responses given to supplemental instructions during the first patient encounter (e.g., if the patient exceeds a threshold number of responses to supplemental instructions, such as 2, 3, 4, or more). In some instances, the determination to prompt the provider to initiate contact with the human interpreter is further based on model 226 and the one or more cultural identifiers for the first patient.

UI module 224 may receive an indication of second user input comprising a selection to initiate contact with the human interpreter. In this instance, UI module 224 may contact the human interpreter, including sending the human interpreter one or more of the first encounter instruction, the first patient response, the supplemental instruction, and the second patient response. Ultimately, UI module 224 may send a record of all or a portion of the encounter between the provider and the patient such that the human interpreter has context for the interaction prior to being introduced into the encounter.

In some instances, prior to receiving the patient information, UI module 224 may output a user interface either under the machine translation platform or the pre-translation platform for patient questions. This may be to initially set which type of patient encounter the first patient is at the facility for, such that encounter module 222 may obtain the proper encounter instructions. For instance, this particular user interface could be integrated into a computing device located at a front desk or a help desk of a medical facility. The patient may initially be asked which language they are comfortable conversing in, and computing device 210 could be configured to output a series of introductory questions, translated into the patient's preferred language, and receive answers to those questions, using either the machine translation platform or the pre-translation platform. Computing device 210 could translate the patient's answers into the language spoken by the healthcare worker, who could further assist the patient. UI module 224 may receive an indication of user input within the user interface indicating a patient introduction. If encounter module 222 is able to retrieve encounter instructions based on that patient introduction, encounter module 222 may enter the pre-translation platform for the determined type of patient encounter.

In some examples, encounter module 222 may further receive patient information for a second patient different than the first patient. Encounter module 222 may determine, based at least in part on the patient information for the second patient and model 226, one or more cultural identifiers for the second patient. The one or more cultural identifiers for the second patient are different than the one or more cultural identifiers for the first patient, indicating that the second patient has a different background than the first patient. Encounter module 222 may retrieve a set of one or more encounter instructions for a second patient encounter for the second patient based on a patient encounter type of the second patient encounter, but the patient encounter type of the second patient encounter is a same type as the patient encounter type of the first patient encounter. In other words, the first patient and the second patient may both be going to a medical facility for generally the same procedure, examination, or process. Encounter module 222 may develop a second updated set of one or more encounter instructions for the second patient encounter by altering the set of one or more encounter instructions for the second patient encounter based on the one or more cultural identifiers for the second patient. Since the cultural indicators for the second patient are different than those of the first patient, the second updated set of one or more encounter instructions is different than the updated set of one or more encounter instructions for the first patient encounter, as the different cultural indicators may necessitate different questions or orders to occur during the encounter. UI module 224 may output, via UIC 112 or output components 246, at least a portion of the second updated set of one or more encounter instructions to guide the second patient encounter.

For example, two individuals may both be visiting a medical clinic for a general physical examination. A first individual may be an Arabic male in a particular city, and model 226 may indicate that Arabic males in that city have a higher prevalence of diabetes than those who are not both Arabic and male in that city. As such, encounter module 222 may add particular encounter instructions to the general physical examination encounter instructions to ensure that diabetes is explicitly checked for or that questions directed to diabetes symptoms or causes are added to the physical examination, when those questions may not be present for other individuals. A second individual may have a born gender of female (e.g., they were born with female reproductive organs) but may have a personal gender of being a male (e.g., they identify as a male). While the second individual may not receive encounter instructions directed at diabetes, like the first individual, encounter module 222 may add encounter instructions to the updated set of encounter instructions specifically directed to hormone therapy, mental health, or gender-based surgeries.

In some examples model 226 is a rule-based model. In other instances, model 226 is an artificial intelligence model. Encounter module 222 may initially train model 226 with data including one or more of country metrics, platform input, cultural markers, government created health data, religious practices, World Health Organization data, client data, public data from one or more public sources, private data from one or more private sources, and company-specific surveys. Encounter module 222 may also update model 226 based on updates to the data listed above, as well as based on one or more patient responses to the updated set of one or more encounter instructions for the first, and any subsequent, patient encounter.

Ultimately, the purpose for encounter module 222 developing model 226 is to identify, for at least a first population of patients each having a first set of one or more cultural identifiers, that a prevalence of a medical tendency within the first population has a difference with a prevalence of the medical tendency within a second population that is statistically significant. A medical tendency may be any medically related disease, malady, deficiency, action, or common assumption that is more prevalent in one population than in another. For instance, people with a first set of cultural indicators may be more prone to heart disease, people with a second set of cultural indicators may be more prone to not following prescription instructions completely, people with a third set of cultural indicators may be less likely to have health insurance, and people with a fourth set of cultural indicators may be celebrating a particular holiday where food is served that does not fit with the individual's personal health characteristics (e.g., those of Jewish faith eating a dairy heavy diet around their holidays while also being lactose intolerant). The difference being statistically significant may mean that the difference meets a threshold, such as a percentage distance (e.g., the prevalence in the first population is X % higher than the prevalence in the second population, with X being a statistically significant marker, such as 1%, 2%, 5%, 10%, or any other percentage deemed to be statistically significant) or a scalar distance (e.g., the prevalence in the first population has X more cases than the prevalence in the second population).

In such instances, in developing the updated set of one or more encounter instructions, encounter module 222 may develop, based at least in part on model 226 and the identified medical tendency, the updated set of one or more encounter instructions. For instance, model 226 may show that the first patient, with their particular cultural indicators, may be more likely to be a smoker. As such, encounter module 222 may, after determining this tendency from model 226, add additional questions to the updated set of one or more encounter instructions focusing on smoking and providing instructions for smoking cessation.

In some examples, encounter module 222 may remove one or more encounter instructions from the updated set of one or more encounter instructions based at least in part on the patient information. For instance, encounter module 222 may determine that an encounter instruction for the updated set of one or more encounter instructions is to ensure that the patient has insurance of some sort, or that the patient is at a higher risk for diabetes. However, if the patient information shows that the first patient has accurate and sufficient health insurance or that the physical measurements of the user make the risk for diabetes lesser than for others in that same population, encounter module 222 may remove the instructions directed to those medical tendencies.

Further, in accordance with the techniques described herein, language module 220 may enable computing device 210 to act purely as a translation platform rather than altering the encounter instructions. For instance, UI module 224 may output, via UIC 212 or output components 246, a human language translation of a first encounter instruction of a set of one or more encounter instructions to guide a first patient encounter with a first patient, with the human language translation being a pre-defined translation. UI module 224 may receive a first patient response to the first encounter instruction. Encounter module 222 may determine, based at least in part on one or more characteristics of the first patient response, to switch from a pre-defined translation platform to a machine translation platform. UI module 224 may receive an indication of user input comprising a supplemental instruction input by the provider, and UI module 224 may output, via UIC 212 or output components 246, a machine translation of the supplemental instruction during the first patient encounter.

In this way, the techniques of this disclosure effect particular treatments and prophylaxes for diseases and medical conditions by utilizing various data points to guide patient encounters. For instance, if a particular malady is more prevalent in groups of people with particular cultural identifiers, the system may automatically recognize when a patient fits into those particular cultural identifiers and alter a set of encounter instructions to ensure that the provider asks questions or performs tests to account for that particular malady. The system may further adjust wording so as not to offend the patient that may be sensitive to particular wording or add further information to discharge instructions. In any case, the system may directly alter how a provider treats a patient during a patient encounter by ensuring that risk factors inherent in patients with certain cultural identifiers are accounted for.

Furthermore, by automatically detecting when to switch platforms between pre-translation platforms (e.g., a platform where questions have previously been translated to account for cultural differences and regional dialects), machine translations (e.g., a real-time translation platform where questions and responses are input in one language and translated by one or more processors into a different language), and an interpreter platform (e.g., where a third-part interpreter is summoned to interpret a conversation between a patient and a provider), the techniques of this disclosure may further effect particular treatments and prophylaxes for diseases and medical conditions by ensuring that accurate and complete information is transferred between the provider and the patient. Furthermore, by automatically detecting when a platform switch is necessary, the techniques of this disclosure may reduce the amount of user inputs provided to the system, thereby improving the computing device overall by reducing the number of physical, logical, or otherwise user-generated inputs received and processed at the computing device that implements these techniques.

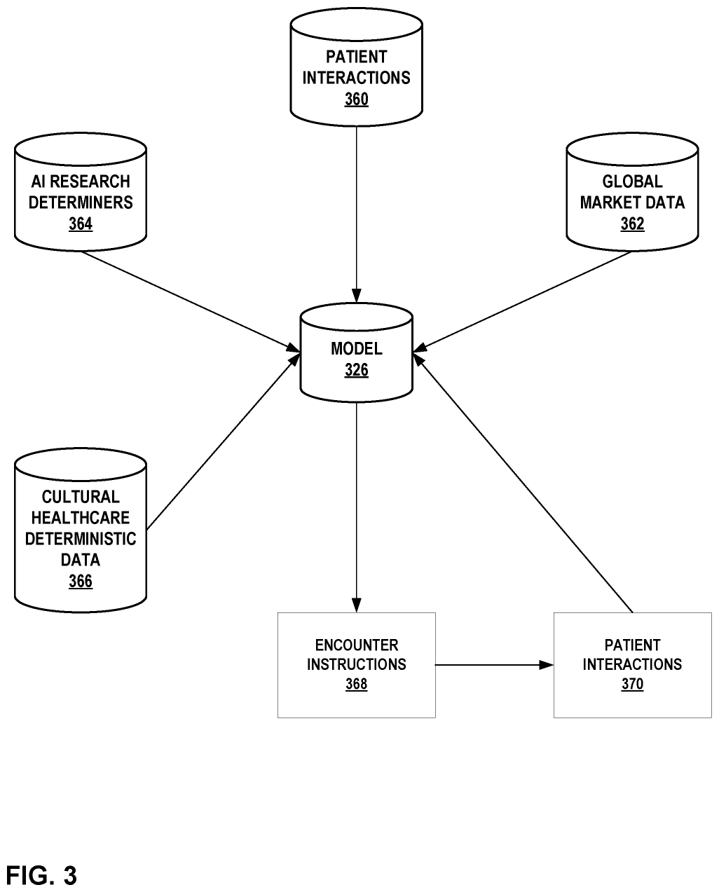

FIG. 3 is a conceptual diagram illustrating model and 326 a plurality of data sources (e.g., 360-366 and 370) that may be used to train a model in accordance with one or more techniques described herein. Model 326 may initially be trained using data from a number of sources, including prior patient interactions data store 360, global market data store 362, AI research determiners data store 364, and cultural healthcare deterministic data 366.

Based on this initial training, a computing device, utilizing model 326, may alter a set of default encounter instructions and output encounter instructions 368 for a particular patient encounter. Throughout the patient encounter, the computing device may receive indications of patient interactions 370, which include responses to various encounter instructions 368. Patient interactions 370 may be further sent to the computing device responsible for maintaining model 326, and the computing device may update model 326 based on patient interactions 370.

Additionally, if the computing device determines that there are updates to outside data points, the computing device may re-access those data points to update and maintain model 326. For instance, if an entity updates any of prior patient interactions data store 360, global market data store 362, AI research determiners data store 364, and cultural healthcare deterministic data 366, the computing device may re-access any of prior patient interactions data store 360, global market data store 362, AI research determiners data store 364, and cultural healthcare deterministic data 366, retrieve the updated data, and update model 326 based on that updated data such that model 326 is routinely up-to-date with the various medical abnormalities that affect particular cultural populations.

In addition to updating model 326, a computing device may send encounter instructions 368 and patient interactions 370 to a printer such that a healthcare provider can receive a hard copy of the encounter to update the patient's medical records. In other instances, the computing device may communicate directly with an electronic medical record (EMR) system through API connectivity (or any other wired or wireless connection) to update the patient's record in the EMR system based on encounter instructions 368 and patient interactions 370. In this way, the computing device that implements the techniques described herein may provide assistance for the entirety of the patient encounter, guiding the encounter from initial check-in through the encounter itself and ending with an assisted (or automatic) update of the patient's medical record.

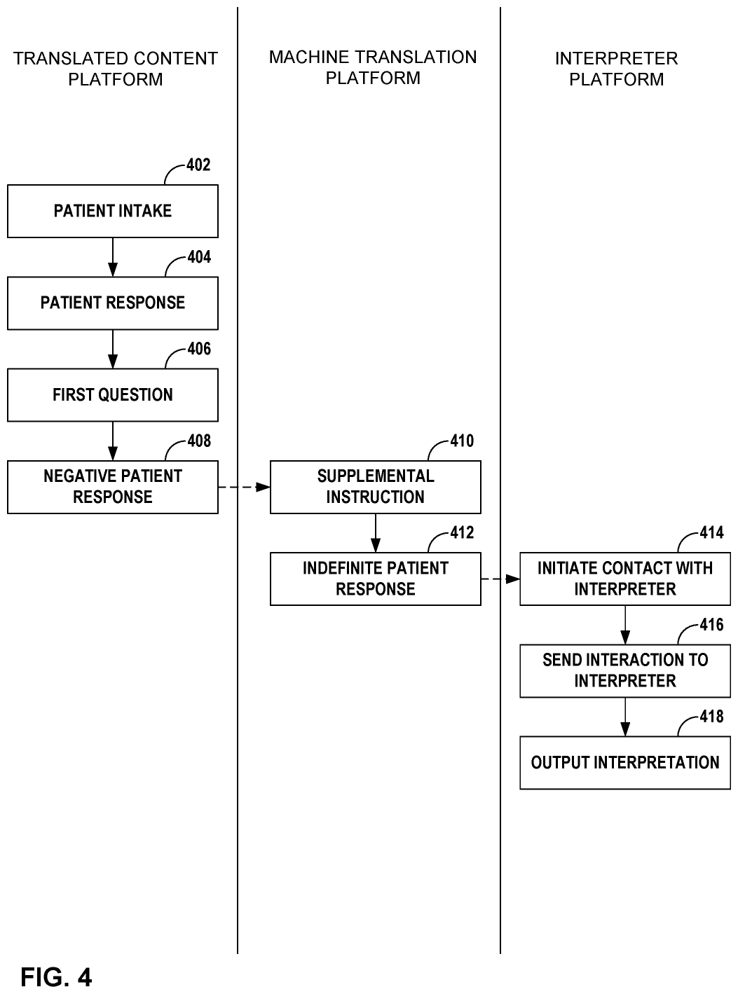

FIG. 4 is a flow diagram illustrating an example platform switching process in accordance with one or more techniques described herein. The techniques of FIG. 4 may be performed by one or more processors of a computing device, such as environment 100 of FIG. 1 and/or computing device 210 illustrated in FIG. 2. For purposes of illustration only, the techniques of FIG. 4 are described within the context of computing device 210 of FIG. 2, although computing devices having configurations different than that of computing device 210 may perform the techniques of FIG. 4.

In accordance with the techniques described herein, language module 220 and encounter module 222 may begin operating in the translated content platform. Encounter module 222 may perform a patient intake process (402) and receive a patient response (404) as part of that intake process. UI module 224 may output a first question (406) and receive a patient response that encounter module 222, after analyzing the response, deems to be negative (e.g., an indication that the patient does not have their insurance card on them) (408).

Language module 220, in response to receiving the negative patient response, may switch the operation of computing device 210 to be under the machine translation platform, where UI module 224 outputs a prompt for a free-form, supplemental instruction from the provider. Language module 220 may output the translated supplemental instruction (410). If encounter module 222 determines that the patient response to that supplemental instruction is valid, encounter module 222 or language module 220 may switch computing device 210 back to the translated content platform, where encounter module 222 may resume the normally performed encounter. If, however, encounter module 222 or language module 220 determines that the patient response is negative (e.g., an indication that the patient does not have health insurance at all) or indefinite (e.g., an indication that the patient does not know if they have health insurance), language module 220 may initiate contact with an interpreter (414) under the interpreter platform. As part of this initiation, encounter module 222 may send a record of the interaction thus far to the interpreter (416). UI module 224 may then output interpretations to and from the interpreter to complete the encounter (418).

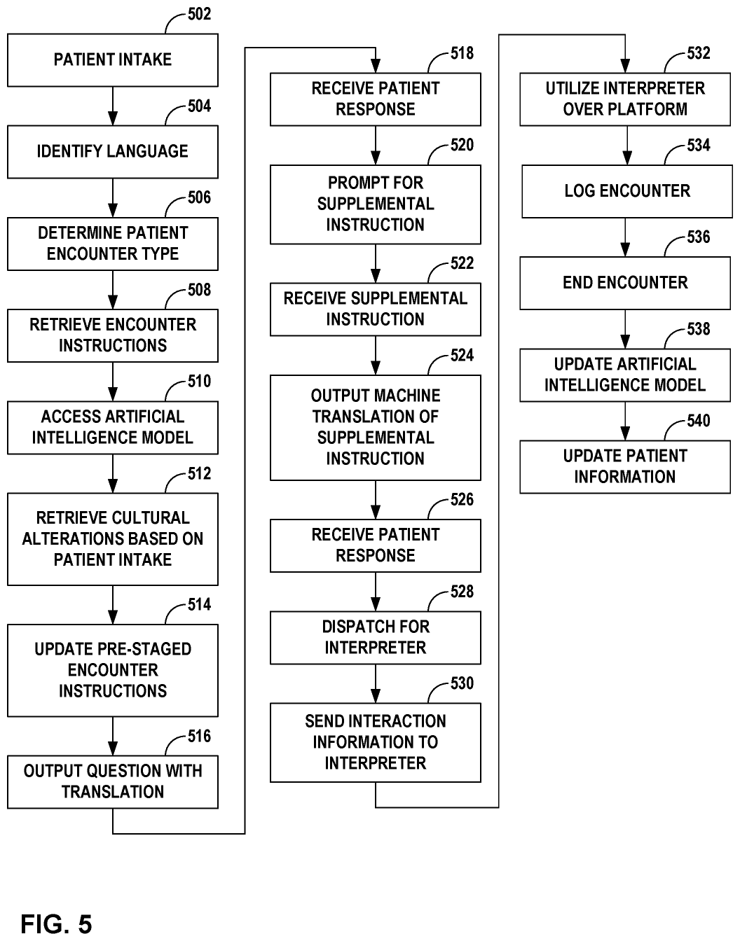

FIG. 5 is a flow diagram illustrating an example patient encounter in accordance with one or more techniques described herein. The techniques of FIG. 5 may be performed by one or more processors of a computing device, such as environment 100 of FIG. 1 and/or computing device 210 illustrated in FIG. 2. For purposes of illustration only, the techniques of FIG. 5 are described within the context of computing device 210 of FIG. 2, although computing devices having configurations different than that of computing device 210 may perform the techniques of FIG. 5.

In accordance with the techniques described herein, encounter module 222 may perform an initial patient intake process (502). Language module 220 may receive the information from that intake process and identify a language that computing device 210 should use for the remainder of the patient encounter (504).

Encounter module 222 may further, based on the patient information from that intake process, determine a patient encounter type for the current patient encounter (e.g., a patient intake process, a medical examination, a pharmaceutical consultation, a follow-up examination, a patient discharge, a patient admittance, in-room patient care, or an unplanned patient visit, among other things) (506). Encounter module 222 may retrieve encounter instructions for the particular type of encounter (508), where the encounter instructions are a basic set of questions, orders, procedures, and/or tests to be run based on the particular type of patient encounter.

Encounter module 222 may then access model 226 (510) and, based on the one or more cultural indications of the patient as determined from that patient's intake information, retrieve cultural alterations to the encounter instructions (512). Encounter module 222 may update the initial encounter instructions to include new encounter instructions, remove old encounter instructions, or alter encounter instructions based on the cultural indications (514).

Language module 220 may translate, and UI module 224 may output, an initial question from the encounter instructions for the patient encounter (516). Encounter module 222 may receive a patient response to that initial question (518). After determining the patient response to be insufficient, encounter module 222 and language module 220 may switch to the machine translation platform, prompting the provider for a supplemental instruction (520). Encounter module 222 may receive the supplemental instruction (522), and language module 220 may perform a machine translation on the supplemental instruction, which UI module 224 outputs (524).

Encounter module 222 receives a patient response to this supplemental instruction (526). Encounter module 222 may deem the response inadequate and dispatch for an interpreter (528). Encounter module 222 may also send interaction information describing the patient encounter thus far (including a transcript of the encounter) to the interpreter (530). Encounter module 222 may utilize the interpreter over the interpreter platform for the remainder of the patient encounter (532), logging the interactions that take place for the remainder of the patient encounter (534).

After ending the encounter (536), encounter module 222 may take the interactions and update model 226 with the various instructions, responses, and sequences of the patient encounter (538). This can help model 226 better provide alterations in the future for other patient encounters. Encounter module 222 may further update patient information for the particular patient such that certain encounter instructions can be ignored in the future due to the patient's provided information (540)

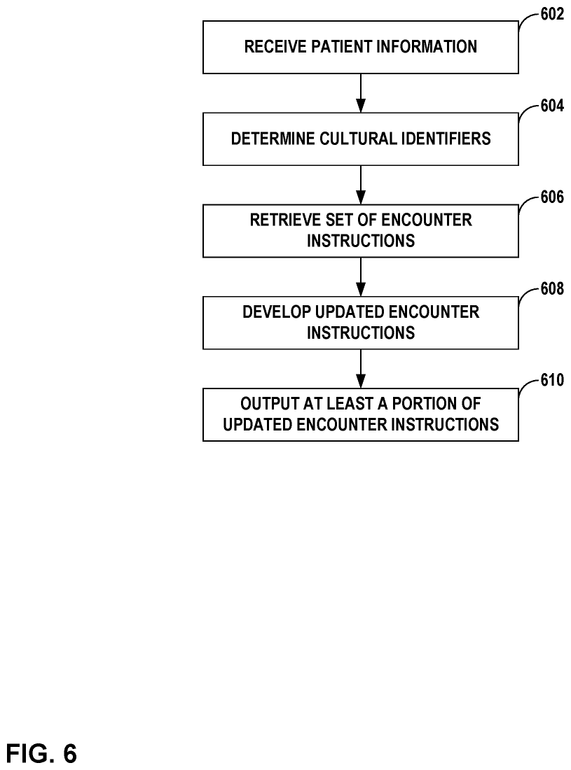

FIG. 6 is a flow diagram illustrating an example patient encounter in accordance with one or more techniques described herein. The techniques of FIG. 6 may be performed by one or more processors of a computing device, such as environment 100 of FIG. 1 and/or computing device 210 illustrated in FIG. 2. For purposes of illustration only, the techniques of FIG. 6 are described within the context of computing device 210 of FIG. 2, although computing devices having configurations different than that of computing device 210 may perform the techniques of FIG. 6.

In accordance with the techniques described herein, encounter module 222 receives patient information for a first patient (602). Encounter module 222 determines, based at least in part on the patient information for the first patient and model 226, one or more cultural identifiers for the first patient (604). Encounter module 222 retrieves a set of one or more encounter instructions for a first patient encounter for the first patient based on a patient encounter type of the first patient encounter for the first patient (606). Encounter module 222 develops an updated set of one or more encounter instructions for the first patient encounter by altering the set of one or more encounter instructions for the first patient encounter based on the one or more cultural identifiers for the first patient (608). UI module 224 outputs, via UIC 212 or output components 246, at least a portion of the updated set of one or more encounter instructions to guide the first patient encounter (610).

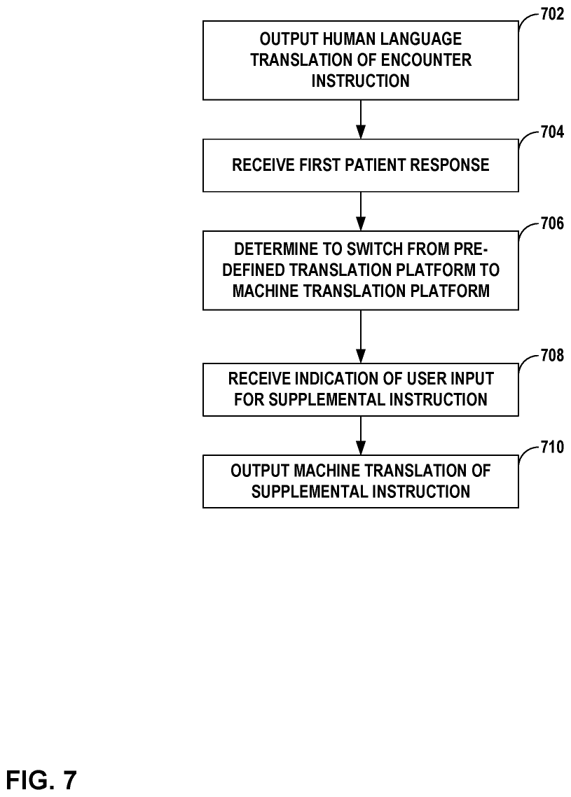

FIG. 7 is a flow diagram illustrating an example platform switching process in accordance with one or more techniques described herein. The techniques of FIG. 7 may be performed by one or more processors of a computing device, such as environment 100 of FIG. 1 and/or computing device 210 illustrated in FIG. 2. For purposes of illustration only, the techniques of FIG. 7 are described within the context of computing device 210 of FIG. 2, although computing devices having configurations different than that of computing device 210 may perform the techniques of FIG. 7.

In accordance with the techniques described herein, UI module 224 outputs, via UIC 212 or output components 246, a human language translation of a first encounter instruction of a set of one or more encounter instructions to guide a first patient encounter with a first patient, where the translation is a pre-defined translation (702). Encounter module 222 receives a first patient response to the first encounter instruction (704). Encounter module 222 determines, based at least in part on one or more characteristics of the first patient response, to switch from a pre-defined translation platform to a machine translation platform (706). Encounter module 222 receives an indication of user input comprising a supplemental instruction input by the provider (708). UI module 224 outputs, via UIC 212 or output components 246, a machine translation of the supplemental instruction during the first patient encounter (710).

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

Various examples of the disclosure have been described. Any combination of the described systems, operations, or functions is contemplated. These and other examples are within the scope of the following claims.

What is claimed is:

1. A method for guiding a patient encounter, the method comprising:
receiving, by one or more processors of a computing device, patient information for a first patient;
determining, by the one or more processors and based at least in part on the patient information for the first patient and a model, one or more cultural identifiers for the first patient;
retrieving, by the one or more processors, a set of one or more encounter instructions for a first patient encounter for the first patient based on a patient encounter type of the first patient encounter for the first patient;
developing, by the one or more processors, an updated set of one or more encounter instructions for the first patient encounter by altering the set of one or more encounter instructions for the first patient encounter based on the one or more cultural identifiers for the first patient; and
outputting, by the one or more processors and via an output component, at least a portion of the updated set of one or more encounter instructions to guide the first patient encounter.

2. The method of claim 1, wherein each encounter instruction in the updated set of one or more encounter instructions comprises one or more of a question to be asked to the first patient by a provider, an order to be given to the first patient by the provider, a procedure to be performed on the first patient by the provider, information to be gathered from the first patient by the provider, or medication to be given to the first patient by the provider.

3. The method of claim 1, further comprising:
outputting, by the one or more processors, a translation of a first encounter instruction of the updated set of one or more encounter instructions, wherein a language for the translation is based on the one or more cultural identifiers for the first patient.

4. The method of claim 3, wherein the translation of the first encounter instruction comprises a pre-translated version of the first encounter instruction presented over a pre-translation platform, wherein the method further comprises:
receiving, by the one or more processors, a first patient response to the first encounter instruction;
determining, by the one or more processors, and based at least in part on one or more characteristics of the first patient response, to switch from the pre-translation platform to a machine translation platform, wherein the one or more characteristics of the first patient response comprise one or more of a time it took for the first patient to provide the first patient response to the first encounter instruction, content of the first patient response, or the first patient response being an indication of silence;
receiving, by the one or more processors, an indication of user input comprising a supplemental instruction input by a provider; and
outputting, by the one or more processors and via the output component, a machine translation of the supplemental instruction during the first patient encounter.

5. The method of claim 4, further comprising, responsive to determining to switch from the pre-translation platform to the machine translation platform, prompting, by the one or more processors, the provider for the supplemental instruction.

6. The method of claim 4, further comprising:
receiving, by the one or more processors, a second patient response to the supplemental instruction;
determining, by the one or more processors, and based at least in part on one or more characteristics of the second patient response, to prompt the provider to initiate contact with a human interpreter, wherein the one or more characteristics of the second patient response comprise one or more of a time it took for the first patient to provide the second patient response to the supplemental instruction, content of the second patient response, the second patient response being an indication of silence, or a number of patient responses given to supplemental instructions during the first patient encounter;
receiving, by the one or more processors, an indication of second user input comprising a selection to initiate contact with the human interpreter; and
contacting, by the one or more processors, the human interpreter, wherein contacting the human interpreter includes sending the human interpreter one or more of the first encounter instruction, the first patient response, the supplemental instruction, and the second patient response.

7. The method of claim 6, wherein determining to switch from the pre-translation platform to the machine translation platform is further based on the model and the one or more cultural identifiers for the first patient, and wherein determining to prompt the provider to initiate contact with the human interpreter is further based on the model and the one or more cultural identifiers for the first patient.

8. The method of claim 4, further comprising:
prior to receiving the patient information, outputting, by the one or more processors, a user interface under the machine translation platform;
receiving, by the one or more processors, an indication of user input within the user interface indicating a patient introduction; and
switching, by the one or more processors, from the machine translation platform to the pre-translation platform for the patient encounter.

9. The method of claim 4, further comprising:
receiving, by the one or more processors, a second patient response to the supplemental instruction;
determining, by the one or more processors, and based at least in part on one or more characteristics of the second patient response, to switch from the machine translation platform to the pre-translation platform; and
outputting, by the one or more processors and via the output component, a pre-translated translation of a second encounter instruction from the updated set of one or more encounter instructions during the first patient encounter.

10. The method of claim 1, wherein outputting at least the portion of the updated set of one or more encounter instructions comprises outputting, by the one or more processors and via the output component, a translation of at least the portion of the updated set of one or more encounter instructions, wherein the translation is based at least in part on the one or more cultural identifiers for the first patient, and
wherein the output component comprises one or more of a screen configured to output one or more of graphical text, videos, or images, a speaker configured to output audio, and a printer configured to print the updated set of one or more encounter instructions.

11. The method of claim 1, wherein the patient encounter type comprises one or more of a patient intake process, a medical examination, a pharmaceutical consultation, a follow-up examination, a patient discharge, a patient admittance, in-room patient care, and an unplanned patient visit.

12. The method of claim 1, wherein the one or more cultural identifiers comprise one or more of a country of origin, a preferred language, a region of origin, a religion, a time of year, a day of a week, an age, a family descendance, a birth gender, a personal gender, a sexual orientation, a skin color, a residence location, or any other culturally descriptive information of the first patient.

13. The method of claim 1, wherein altering the set of one or more encounter instructions comprise adding a new encounter instruction to the set of one or more encounter instructions, removing an encounter instruction from the set of one or more encounter instructions, or changing content of an encounter instruction from the set of one or more encounter instructions.

14. The method of claim 1, further comprising:
receiving, by the one or more processors, an indication of user input comprising a patient response to a first encounter instruction of the updated set of one or more encounter instructions; and
translating, by the one or more processors, the patient response into a language spoken by a provider.

15. The method of claim 1, further comprising:
receiving, by the one or more processors, patient information for a second patient;
determining, by the one or more processors and based at least in part on the patient information for the second patient and the model, one or more cultural identifiers for the second patient, wherein the one or more cultural identifiers for the second patient are different than the one or more cultural identifiers for the first patient;
retrieving, by the one or more processors, a set of one or more encounter instructions for a second patient encounter for the second patient based on a patient encounter type of the second patient encounter, wherein the patient encounter type of the second patient encounter is a same type as the patient encounter type of the first patient encounter;
developing, by the one or more processors, a second updated set of one or more encounter instructions for the second patient encounter by altering the set of one or more encounter instructions for the second patient encounter based on the one or more cultural identifiers for the second patient, wherein the second updated set of one or more encounter instructions is different than the updated set of one or more encounter instructions for the first patient encounter; and
outputting, by the one or more processors and via the output component, at least a portion of the second updated set of one or more encounter instructions to guide the second patient encounter.

16. The method of claim 1, wherein the model comprises an artificial intelligence model, wherein the method further comprises:
initially training, by the one or more processors, the artificial intelligence model with data comprising one or more of country metrics, platform input, cultural markers, government created health data, religious practices, World Health Organization data, client data, public data from one or more public sources, private data from one or more private sources, and company-specific surveys; and
updating, by the one or more processors, the artificial intelligence model based on updates to the data and one or more patient responses to the updated set of one or more encounter instructions.

17. The method of claim 16, further comprising:
developing, by the one or more processors, the artificial intelligence model to identify, for at least a first population of patients each having a first set of one or more cultural identifiers, that a prevalence of a medical tendency within the first population of patients has a difference with a prevalence of the medical tendency within a second population of patients that is statistically significant,
wherein the difference being statistically significant comprises the difference meeting a threshold, wherein the threshold comprises one or more of a percentage distance or a scalar distance, and
wherein developing the updated set of one or more encounter instructions comprises developing, based at least in part on the artificial intelligence model and the medical tendency, the updated set of one or more encounter instructions.

18. The method of claim 1, further comprising:
removing, by the one or more processors, one or more encounter instructions from the updated set of one or more encounter instructions based at least in part on the patient information.

19. A system comprising:
a data store configured to store at least an artificial intelligence model, patient information for a plurality of patients, and a plurality of sets of one or more encounter instructions for patient encounters;
an output component; and
one or more processors configured to:
receive patient information for a first patient of the plurality of patients;
determine, based at least in part on the patient information for the first patient and the artificial intelligence model, one or more cultural identifiers for the first patient;
retrieve, from the data store, a first set of one or more encounter instructions for a first patient encounter for the first patient based on a patient encounter type of the first patient encounter for the first patient;
develop an updated set of one or more encounter instructions for the first patient encounter by altering the first set of one or more encounter instructions for the first patient encounter based on the one or more cultural identifiers for the first patient; and
output, via the output component, at least a portion of the updated set of one or more encounter instructions to guide the first patient encounter.

20. A method for assisting a patient encounter, the method comprising:
outputting, by one or more processors of a computing device and via an output component, a human language translation of a first encounter instruction of a set of one or more encounter instructions to guide a first patient encounter with a first patient, wherein the human language translation is a pre-defined translation;
receiving, by the one or more processors, a first patient response to the first encounter instruction;
determining, by the one or more processors, and based at least in part on one or more characteristics of the first patient response, to switch from a pre-defined translation platform to a machine translation platform;
receiving, by the one or more processors, an indication of user input comprising a supplemental instruction input by a provider; and
outputting, by the one or more processors and via the output component, a machine translation of the supplemental instruction during the first patient encounter.
